(19) United States
(12) Patent Application Publication
(10) Pub. No.: US 2004/0120916 A1
(43) Pub. Date: Jun. 24, 2004

(54) Contact lens care compositions, methods of use and preparation which protect ocular tissue membrane integrity

(75) Inventor: Stan Huth, Newport Beach, CA (US)

Correspondence Address: Nicole S. Bradley, PILLSBURY WINTHROP LLP, Suite 2800, 725 South Figueroa Street, Los Angeles, CA, 90017, US

(73) Assignee: ADVANCED MEDICAL OPTICS, INC.

(21) Appl. No.: 10/328,641

(22) Filed: Dec. 23, 2002

(51) Int. Cl. A61K 31/716; A61K 31/185

(52) U.S. Cl. 424/70.13; 514/57; 514/553

(57) ABSTRACT

A multi-purpose contact lens care solution comprising taurine, a liquid aqueous medium, an antimicrobial component, a surfactant and a buffer. This solution prevents losses in ocular tissue membrane integrity during contact lens wear.

### BACKGROUND OF THE INVENTION

1. Area of the Art

The present invention relates to compositions and methods for contact lens care. More particularly, the invention relates to contact lens care compositions comprising taurine which provide a cell membrane protection function for ocular tissue cells during contact lens wear.

2. Description of the Prior Art

Contact lens wear induces adverse changes in ocular tissues and the tear film. These changes include cornea lactic acidosis and subsequent cornea swelling as a consequence of hypoxia induced by low oxygen gas transmission, changes in corneal epithelial tissue thickness, changes in corneal epithelial and endothelial cell morphology, epithelial surface cell exfoliation, hyperemia (red eye), adverse changes in corneal and conjunctival cell membrane integrity and destabilization of the tear film. Changes in cell membrane integrity can be measured clinically via measurements of lactate dehydrogenase enzyme release, fluorescein barrier permeability or other methods. Corneal epithelial cell membrane integrity is believed to be critical to maintain a tissue barrier function to prevent ocular infection.

Adverse changes in ocular tissues during contact lens wear also may arise due to exposure of ocular tissues to preservatives, disinfecting agents, cleaning agents and other components in the contact lens care solutions. This can occur through tissue contact with solutions which may directly contact ocular tissues during application or tissue contact with solutions which may adsorb or absorb to the contact lens during treatment of the contact lens by the solution, and subsequently desorb from the contact lens during wear into the eye.

Contact lens solutions have become complex formulations of multiple components which provide several functions. Attempts have been made to ameliorate the adverse effects of contact lenses and contact lens care solutions on ocular tissues, with mixed results. The best examples of success in changing contact lens care solutions to ameliorate their adverse effects on ocular tissues is represented by the creation of polymeric contact lens disinfecting agents, antimicrobial systems which do not bind to contact lens surfaces and the inclusion of water-soluble polymers and electrolytes such as potassium chloride, magnesium and calcium chloride into contact lens multi-purpose and rewetting solutions. However, despite these favorable changes in the compositions of contact lens care solutions, none provide perfect in-eye performance without some measure of adverse effect on ocular tissues. Some degree of compromise to the tear film, tissue or cellular membrane integrity, such as corneal epithelial cell membrane integrity, remains with all current contact lens care solutions.

The classic clinical symptoms of allergic conjunctivitis [type I] allergy—itching and lacrimation (tearing)—are the effect of histamine: Tears from asymptomatic and symptomatic contact lens wearers are reported to have significant levels of histamine, although a clear correlation between histamine level and adverse ocular response during contact lens wear is lacking (Aust N Z J Ophthalmol 1997 May;25 Suppl 1: S27-9). A study was recently published of the cytoprotective effect of amino acids, including taurine, on local toxicity caused by sodium laurate, a drug absorption enhancer, in rat large-intestinal tissue (J Pharm Sci 2002 March;91(3):730-43). This study showed that although sodium laurate stimulated the release of histamine from rat large-intestinal tissue, amino acids including taurine were found to suppress the release of histamine enhanced by sodium laurate.

Another recent study examined the protective effects of several amino acids including taurine on gastric hemorrhagic erosions in acid-irrigated stomachs of lipopolysaccharide (LPS)-intoxicated rats (Chin J Physiol 1999 Sep. 30;42(3):161-9). Ulcerogenic parameters including mucosal histamine concentrations were markedly enhanced in LPS rat stomachs irrigated with acid solution. Taurine caused dose-dependent attenuations of these ulcerogenic parameters in LPS rats.

Taurine also significantly suppressed the decrease in the transepithelial electrical resistance, a measure of cell membrane integrity, caused by sodium laurate in rat large-intestinal tissue in the aforementioned study. More recently, a study of cell survival during a 450 m0sm/kg hypertonic medium challenge was conducted on human corneal epithelial cells (Shioda et al., IOVS, September 2002, Vol. 43, No. 9, pp. 2916-2922). This study showed that additional medium supplementation with 1 mM taurine (0.0125 w/v %) significantly increased cell survival. The authors concluded that the results reflected an antioxidant or membrane stabilization effect of taurine. This study did not disclose, hint, suggest or otherwise direct anyone skilled in the art to the instant teachings or that taurine could provide a protective benefit during contact lens wear.

Taurine has previously been included in contact lens care compositions. For example, Kawai, et al., in U.S. Pat. No. 5,302,312, disclose the use of taurine as a water-soluble component of a detergent cleaning composition. The detergent cleaning composition also comprises a pasting agent composed of a copolymer of polyhydric alcohol and a cross-linked acrylic acid. This composition is designed to be rinsed from the contact lens after use, and not left in the eye.

Huth, in U.S. Pat. No. 5,389,383, discloses methods and compositions for treating hypoxia-associated ocular complications during contact lens wear. The compositions and associated methods taught by the '383 patent may comprise at least two agents selected from the group consisting of a heme oxygenase inducer, a membrane-permeable anti-acidosis buffer and an osmoprotectant. The '383 patent discloses by inference, if not expressly, the possibility that taurine is an osmoprotectant.

Kato, in U.S. Pat. No. 5,945,121, discloses liposome eye drops containing glucose, inorganic salts and taurine which are useful for treating dry eye or mitigating its symptoms. A cellular protective function for taurine is not disclosed.

Shinohara, et al., in U.S. Pat. No. 5,998,488 disclose a method for inhibiting antimicrobial preservative from adsorbing to contact lens, comprising a cationic preservative, a cyclodextrin, ethylenediaminetetraacetic acid, boric acid or borax, and optionally taurine or another agent. However, this reference does not disclose any mechanism or possible role played by taurine in the described method.

Tsuzuki, et al., in U.S. Pat. No. 6,121,327, disclose a contact lens disinfecting solution which does not include protease, and which contains in an aqueous medium, an organic nitrogen disinfectant and 0.01-5 w/v % of at least one polyol, and further contains bis(2-hydroxyethyl) iminotris(hydroxymethyl)methane. To effectively maintain the pH of the contact lens disinfecting solution within the desired range for assuring safety to the eyes, at least one buffer is added. Tsuzuki discloses only that taurine and its derivatives may be used as a buffer, among many others listed.

Thus, none of the aforementioned prior art disclose a contact lens care solution which includes taurine which serves a membrane protective function for ocular tissues. In view of these limitations to contact lens care compositions, it would be advantageous to have contact lens care compositions which better maintain ocular tissue cell membrane integrity during contact lens wear.

### SUMMARY OF THE INVENTION

New compositions for treating contact lenses have been discovered. The present compositions that may be, for example, multi-purpose aqueous solutions, include taurine, antimicrobial components, preferably reduced concentrations of antimicrobial components, in combination with phosphate buffers and viscosity inducing components to provide the desired antimicrobial activity and performance effectiveness and, importantly, substantial, preferably enhanced, lens wearer/user comfort and acceptability benefits. These compositions are surprising and unexpected in view of the above noted prior art which employs relatively large concentrations of antimicrobial components and/or buffering systems other than phosphate buffering systems and/or does not employ viscosity inducing components.

In addition, the inclusion of one or more other components in the present compositions is effective in providing additional beneficial properties to the compositions, and preferably provide further lens wearer/user comfort and acceptability benefits. The present compositions have a multitude of applications, for example, as disinfecting, cleaning, soaking, wetting, rewetting, rinsing, storing, in-the-eye cleaning, and conditioning compositions, for contact lens care, while providing substantial lens wearer/user comfort and acceptability. The present compositions necessarily increase user compliance, that is promote regular and consistent contact lens care, and, ultimately, lead to or facilitate better ocular health.

In one embodiment of the present invention, multi-purpose solutions for contact lens care are provided. Such solutions comprise taurine, an aqueous liquid medium; an antimicrobial component in an amount effective to disinfect a contact lens contacted with the solution; a surfactant in an amount effective in cleaning a contact lens contacted with the solution; a phosphate buffer component in an amount effective in maintaining the pH of the solution within a physiologically acceptable range; a viscosity inducing component present in an effective amount; and a tonicity component in an amount effective in providing the desired tonicity to the solution.

In a further embodiment of the present invention, the multi-purpose solutions for contact lens care include taurine, which prevents losses in ocular tissue membrane integrity during contact lens wear. The taurine is preferably present in an amount in the range of about 0.01% or about 0.5% to about 1.0% or about 2%. The lower limit of taurine concentration is determined by its effectiveness. The upper limit of taurine concentration is determined by the feel of the solution in the eye and/or any potential cytotoxicity.

The antimicrobial component may be any suitable, preferably ophthalmically acceptable, material effective to disinfect a contact lens contacted with the present solutions or alternatively adequately preserve a solution such as a contact lens rewetting solution. Preferably, the antimicrobial component is selected from biguanides, biguanides polymers, salts thereof and mixtures thereof, and is present in an amount in the range of about 0.1 ppm to about 3 ppm or less than 5 ppm (w/v). By way of example, and not of limitation, the antimicrobial component may be a monomeric quaternary ammonium or biguanide compound such as chlorhexidine digluconate, chlorhexidine diacetate, benzethonium chloride and myristamidopropyldimethylamine. The antimicrobial component may also be a polymeric quaternary ammonium compound such as Polyquad.RTM. (polyquatemium-1) or poly [oxyethylene (dimethyliminio)ethylene-(dimethyliminio)ethylene dichloride] (sold under the trademark WSCP by Buckman Laboratories, Inc.). The preferred relatively reduced concentration of the antimicrobial component has been found to be very effective, in the present compositions, in disinfecting contact lenses contacted with the compositions, while at the same time promoting lens wearer/user comfort and acceptability.

Any suitable, preferably ophthalmically acceptable, surfactant component which is effective in cleaning contact lenses may be employed. The surfactant component preferably is non ionic and, more preferably, is selected from poly(oxyethylene)-poly(oxypropylene) block copolymers and mixtures thereof.

Any suitable, preferably ophthalmically acceptable viscosity inducing or thickening agent may be included in the present compositions. The viscosity inducing component preferably is selected from cellulosic derivatives and mixtures thereof and is present in an amount in the range of about 0.05% or about 1.5% to about 3% or about 5.0% (w/v). Without wishing to limit the invention to any particular theory of operation, it is believed that the presence of a viscosity inducing component at least assists in providing the lens wearer/user comfort and acceptability benefits of the present invention, which promote regular and consistent contact lens care and ultimately lead to or facilitate better ocular health. The present combinations of components, for example, including such viscosity inducing components, are effective in providing the degree of lens wearer/user comfort and acceptability benefits described herein.

Although any suitable, necessarily ophthalmically acceptable, tonicity component may be employed, an extremely useful tonicity component is a combination of sodium chloride and potassium chloride.

The present compositions preferably include an effective amount of a chelating component. Any suitable, preferably ophthalmically acceptable, chelating component may be included in the present compositions, although ethylenediaminetetraacetic acid (EDTA), salts thereof and mixtures thereof are particularly effective. More preferably, the present compositions include chelating components in effective amounts less than about 0.05% (w/v) and still more preferably 0.02° s (w/v) or less. Such reduced amounts of chelating component in the present compositions remain effective in providing the desired chelating and/or sequestering functions while, at the same time, are better tolerated in the eye, thereby reducing the risk of user discomfort and/or ocular irritation.

Various combinations of two or more of the above noted components may be used in providing at least one of the benefits described herein. Therefore, each and every such combination is included within the scope of the present invention.

These and other aspects of the present invention are apparent in the following detailed description, examples and claims.

### DETAILED DESCRIPTION

The present compositions have a multitude of applications, for example, as disinfecting, cleaning, soaking, wetting, rewetting, rinsing, storing, in-the-eye cleaning, and conditioning compositions, for contact lens care, while providing substantial lens wearer/user comfort and acceptability. Any contact lenses, for example, conventional hard contact lenses, rigid gas permeable contact lenses and soft, hydrophilic or hydrogel, contact lenses, can be treated in accordance with the present invention.

In one embodiment, the present compositions comprise a liquid aqueous medium; taurine; an antimicrobial component in the liquid aqueous medium in an amount effective to disinfect a contact lens contacted with the composition; a surfactant, usually a non ionic surfactant, component in an amount effective in cleaning a contact lens contacted with the composition; a phosphate buffer component in an amount effective in maintaining the pH of the composition within a physiologically acceptable range; an effective amount of a viscosity inducing component; and an effective amount of a tonicity component. The present compositions preferably include an effective amount of a chelating or sequestering component, more preferably in a range of less than 0.05% (w/v). Each of the components, in the concentration employed, included in the solutions and the formulated solutions of the present invention generally are ophthalmically acceptable. In addition, each of the components, in the concentration employed, included in the present solutions usually is soluble in the liquid aqueous medium.

A solution or component thereof is “ophthalmically acceptable” when it is compatible with ocular tissue, that is, it does not cause significant or undue detrimental effects when brought into contact with ocular tissue. Preferably, each component of the present compositions is also compatible with the other components of the present compositions. The present compositions are more preferably substantially ophthalmically optimized. An ophthalmically optimized composition is one which, within the constraints of component chemistry, minimizes ocular response, or conversely delivers ophthalmic benefit to the lens wearing eye.

The presently useful antimicrobial components include chemicals which derive their antimicrobial activity through a chemical or physiochemical interaction with microbes or microorganisms, such as those contaminating a contact lens. Suitable antimicrobial components are those generally employed in ophthalmic applications and include, but are not limited to, quaternary ammonium salts used in ophthalmic applications such as poly [dimethylimino-2-butene-1,4-diyl] chloride, alpha-[4-tris(2-hydroxyethyl)ammonium]-dichloride (chemical registry number 75345-27-6, available under, the trademark Polyquaternium 1® from Onyx Corporation), benzalkonium halides, and biguanides, such as salts of alexidine, alexidine-free base, salts of chlorhexidine, hexamethylene biguanides and their polymers, and salts thereof, antimicrobial polypeptides, chlorine dioxide precursors, and the like and mixtures thereof. Generally, the hexamethylene biguanide polymers (PHMB), also referred to as polyaminopropyl biguanide (PAPB), have molecular weights of up to about 100,000. Such compounds are known and are disclosed in Ogunbiyi et al, U.S. Pat. No. 4,759,595, the disclosure of which is hereby incorporated in its entirety by reference herein.

Generally, the antimicrobial component is present in the liquid aqueous medium at an ophthalmically acceptable or safe concentration such that the user can remove the disinfected lens from the liquid aqueous medium and thereafter directly place the lens in the eye for safe and comfortable wear. Alternatively, the antimicrobial component is present in the liquid aqueous medium at an ophthalmically acceptable or safe concentration and sufficient for maintaining preservative effectiveness. The antimicrobial components useful in the present invention preferably are present in the liquid aqueous medium in concentrations in the range of about 0.00001% to about 2% (w/v), and more preferably in concentrations in the range of about 0.00005% to about 0.01% (w/v).

The antimicrobial components suitable for inclusion in the present invention include chlorine dioxide precursors. Specific examples of chlorine dioxide precursors include stabilized chlorine dioxide (SCD), metal chlorites, such as alkali metal and alkaline earth metal chlorites, and the like and mixtures thereof. Technical grade sodium chlorite is a very useful chlorine dioxide precursor. Chlorine dioxide containing complexes such as complexes of chlorine dioxide with carbonate, chlorine dioxide with bicarbonate and mixtures thereof are also included as chlorine dioxide precursors. The exact chemical composition of many chlorine dioxide precursors, for example, SCD and the chlorine dioxide complexes, is not completely understood. The manufacture or production of certain chlorine dioxide precursors is described in McNicholas, U.S. Pat. No. 3,278,447, which is incorporated in its entirety herein by reference. Specific examples of useful SCD products include that sold under the trademark Dura Klor® by Rio Linda Chemical Company, Inc., and that sold under the trademark Anthium Dioxide® by International Dioxide, Inc.

If a chlorine dioxide precursor in included in the present compositions, it generally is present in an effective preservative or contact lens disinfecting amount. Such effective preservative or disinfecting concentrations usually are in the range of about 0.002 to about 0.06% (w/v) of the present compositions. Such chlorine dioxide precursors may be used in combination with other antimicrobial components, such as biguanides, biguanide polymers, salts thereof and mixtures thereof.

In the event that chlorine dioxide precursors are employed as antimicrobial components, the compositions usually have an osmolality of at least about 200 mOsmol/kg and are buffered to maintain the pH within an acceptable physiological range, for example, a range of about 6 to about 10.

In one embodiment, the antimicrobial component is non-oxidative. It has been found that reduced amounts of non-oxidative antimicrobial components, for example, in a range of about 0.1 ppm to about 3 ppm or less than 5 ppm (w/v), in the present compositions are effective in disinfecting contact lenses and reduce the risk of such antimicrobial components causing ocular discomfort and/or irritation. Such reduced concentration of antimicrobial component is very useful when the antimicrobial component employed is selected from biguanides, biguanide polymers, salts thereof and mixtures thereof.

When a contact lens is desired to be disinfected by the present compositions, an amount of the antimicrobial component effective to disinfect the lens is used. Generally, such an effective amount of the antimicrobial component reduces the microbial burden or load on the contact lens by one log order in three hours. More preferably, an effective amount of the disinfectant reduces the microbial load by one log order in one hour.

The phosphate buffer component is present in an amount effective to maintain the pH of the composition or solution in the desired range, for example, in a physiologically acceptable range of about 6 to about 9. In particular, the solution has a pH in the range of about 6 to about 8. The phosphate buffer component includes one or more phosphate buffers, for example, combinations of monobasic phosphates, dibasic phosphates and the like. Particularly useful phosphate buffers are those selected from phosphate salts of alkali and/or alkaline earth metals. Examples of suitable phosphate buffers include one or more of sodium dibasic phosphate (Na₂HPO₄) sodium monobasic phosphate (NaH₂PO₄) and potassium monobasic phosphate (KH₂PO₄). The present buffer components frequently are used in amounts in a range of about 0.01% or about 0.02% to about 0.5% (w/v), calculated as phosphate ion.

The present compositions usually further comprise effective amounts of one or more additional components, such as a detergent or surfactant component; a viscosity inducing or thickening component; a chelating or sequestering component; a tonicity component; and the like and mixtures thereof. The additional component or components may be selected from materials which are known to be useful in contact lens care compositions and are included in amounts effective to provide the desired effect or benefit. When an additional component is included, it is generally compatible under typical use and storage conditions with the other components of the composition. For instance, the aforesaid additional component or components are substantially stable in the presence of the antimicrobial and buffer components described herein.

A surfactant component generally is present in an amount effective in cleaning, that is to at least facilitate removing, and preferably effective to remove, debris or deposit material from, a contact lens contacted with the surfactant containing solution. Exemplary surfactant components include, but are not limited to, nonionic surfactants, for example, polysorbates (such as polysorbate 20-Trademark Tween 20), 4-(1,1,3,3-tetramethylbutyl)phenol/poly(oxyethylene) polymers (such as the polymer sold under the trademark Tyloxapol), poly(oxyethylene)-poly(oxypropylene) block copolymers, glycolic esters of fatty acids and the like, and mixtures thereof.

The surfactant component is generally nonionic, and usually is selected from poly(oxyethylene)-poly(oxypxopylene) block copolymers and mixtures thereof. Such surfactant components can be obtained commercially from the BASF Corporation under the trademark Pluronic®. Such block copolymers can be generally described as polyoxyethylene/polyoxypropylene condensation polymers terminated in primary hydroxyl groups. They may be synthesized by first creating a hydrophobe of desired molecular weight by the controlled addition of propylene oxide to the two hydroxyl groups of propylene glycol or glycerin. In the second step of the synthesis, ethylene oxide is added to sandwich this hydrophobe between hydrophile groups.

In accordance with a more preferred embodiment of the invention, such block copolymers having molecular weights in the range of about 2500 to 13,000 daltons are suitable, with a molecular weight range of about 6000 to about 12,000 daltons being still more preferred. Specific examples of surfactants which are satisfactory include: poloxamer 108, poloxamer 188, poloxamer 237, poloxamer 238, poloxamer 288 and poloxamer 407. Particularly good results are obtained poloxamer 237.

The amount of surfactant component, if any, present varies over a wide range depending on a number of factors, for example, the specific surfactant or surfactants being used, the other components in the composition and the like. Often the amount of surfactant is in the range of about 0.005% or about 0.01% to about 0.1% or about 0.5% or about 1.0% (w/v).

The viscosity inducing components employed in the present solutions preferably are effective at low or reduced concentrations, are compatible with the other components of the present solutions and are nonionic. Such viscosity inducing components are effective to enhance and/or prolong the cleaning and wetting activity of the surfactant component and/or condition the lens surface rendering it more hydrophilic (less lipophilic) and/or to act as a demulcent on the eye. Increasing the solution viscosity provides a film on the lens which may facilitate comfortable wearing of the treated contact lens. The viscosity inducing component may also act to cushion the impact on the eye surface during insertion and serves also to alleviate eye irritation.

Suitable viscosity inducing components include, but are not limited to, water soluble natural gums, cellulose-derived polymers and the like. Useful natural gums include guar gum, gum tragacanth and the like. Useful cellulose-derived viscosity inducing components include cellulose-derived polymers, such as hydroxypropyl cellulose, hydroxypropylmethyl cellulose, carboxymethyl cellulose, methyl cellulose, hydroxyethyl cellulose and the like. More preferably, the viscosity inducing agent is selected from cellulose derivatives (polymers) and mixtures thereof. A very useful viscosity inducing component is hydroxypropylmethyl cellulose (HPMC).

The viscosity inducing component is used in an amount effective to increase the viscosity of the solution, preferably to a viscosity in the range of about 1.5 to about 30, or even as high as about 750, cps at 25° C., preferably as determined by USP test method No. 911 (USP 23, 1995). To achieve this range of viscosity increase, an amount of viscosity inducing component of about 0.01% to about 5% (w/v) preferably is employed, with amounts of about 0.05% to about 0.5% being more preferred.

A chelating or sequestering component preferably is included in an amount effective to enhance the effectiveness of the antimicrobial component and/or to complex with metal ions to provide more effective cleaning of the contact lens.

A wide range of organic acids, amines or compounds which include an acid group and an amine function are capable of acing as chelating components in the present compositions. For example, nitrilotriacetic acid, diethylenetriaminepentacetic acid, hydroxyethylethylene-diaminetriacetic acid, 1,2-diaminocyclohexane tetraacetic acid, hydroxyethylaminodiacetic acid, ethylenediamine-tetraacetic acid and its salts, polyphosphates, citric acid and its salts, tartaric acid and its salts, and the like and mixtures thereof, are useful as chelating components. Ethylenediaminetetraacetic acid (EDTA) and its alkali metal salts, are preferred, with disodium salt of EDTA, also known as disodium edetate, being particularly preferred.

The chelating component preferably is present in an effective amount, for example, in a range of about 0.01% and about 1% (w/v) of the solution.

In a very useful embodiment, particularly when the chelating component is EDTA, salts thereof and mixtures thereof, a reduced amount is employed, for example, in the range of less than about 0.05% (w/v) or even about 0.02% (w/v) or less. Such reduced amounts of chelating component have been found to be effective in the present compositions while, at the same time, providing for reduced discomfort and/or ocular irritation.

The liquid aqueous medium used is selected to have no substantial deleterious effect on the lens being treated, or on the wearer of the treated lens. The liquid medium is constituted to permit, and even facilitate, the lens treatment or treatments by the present compositions. The liquid aqueous medium advantageously has an osmolality in the range of at least about 200-mOsmol/kg to about 300 or about 350 mOsmol/kg. The liquid aqueous medium more preferably is substantially isotonic or hypotonic (for example, slightly hypotonic) and/or is ophthalmically acceptable.

The liquid aqueous medium preferably includes an effective amount of a tonicity component to provide the liquid medium with the desired tonicity. Such tonicity components may be present in the liquid aqueous medium and/or may be introduced into the liquid aqueous medium. Among the suitable tonicity adjusting components that may be employed are those conventionally used in contact lens care products, such as various inorganic salts. Sodium chloride and/or potassium chloride and the like are very useful tonicity components. The amount of tonicity component included is effective to provide the desired degree of tonicity to the solution. Such amount may, for example, be in the range of about 0.1% to about 1.5% (w/v). If a combination of sodium chloride and potassium chloride is employed, it is preferred that the weight ratio of sodium chloride to potassium chloride be in the range of about 2.5 to about 6 or about 8.

The amount of taurine useful in the present invention may be determined by objective clinical measures such as tear LDH release from corneal epithelial cells or fluorescein barrier permeability measurements or another means to evaluate ocular cell membrane integrity such as fluorescein or rose bengal staining. Yet another means to evaluate ocular cell membrane integrity is the use of confocal microscopy to measure epithelial cell area. In lieu of using tear LDH as a response factor, another inflammatory mediator may be measured in tears to indicate a beneficial effect from taurine. Useful amounts of taurine can also be determined by subjective clinical measures such as itching, lacrimation (tearing) and comfort. The amount of taurine useful in the present invention is generally from about 0.01 to about 2.0 w/v %. The preferred amount is 0.05 to 1.00 w/v %.

Methods for treating a contact lens using the herein described compositions are included within the scope of the invention. Such methods comprise contacting a contact lens with such a composition at conditions effective to provide the desired treatment to the contact lens.

The contacting temperature is preferred to be in the range of about 0° C. to about 100° C., and more preferably in the range of about 10° C. to about 60° C. and still more preferably in the range of about 15° C. to about 30° C. Contacting at or about ambient temperature is very convenient and useful. The contacting preferably occurs at or about atmospheric pressure. The contacting preferably occurs for a time in the range of about 5 minutes or about 1 hour to about 12 hours or more.

The contact lens can be contacted with the liquid aqueous medium by immersing the lens in the medium. During at least a portion of the contacting, the liquid medium containing the contact lens can be agitated, for example, by shaking the container containing the liquid aqueous medium and contact lens, to at least facilitate removal of deposit material from the lens. After such contacting step, the contact lens may be manually rubbed to remove further deposit material from the lens. The cleaning method can also include rinsing the lens substantially free of the liquid aqueous medium prior to returning the lens to a wearer's eye.

The following examples, while not limiting, are illustrative of the invention.

### EXAMPLE 1

Formulations C1, C2 and C3 in Table 1 were clinically evaluated in a 90 day contact lens wear study. Opti-free® Express® multi-purpose solution was evaluated as a fourth solution in this study (Opti-free® Express® is denoted by “O-F” herein). Opti-free® Express® contains a different antimicrobial agent and other excipients which are not present in any of the C1-C3 formulas and in addition does not contain taurine. There were approximately 70 subjects in each of the four test groups of the clinical study, one group per solution.

TABLE 1
Formulations For Clinical Evaluation

| | Formulations | | |
|---|---|---|---|
| | 9450X | 9451X | 945X |
| Ingredients | Normal | Normal | High |
| (all | PHMB | PHMB | PHMB |
| concentrations | Glycol | Glycol + Taurine | Glycol + Taurine |
| in v/w %) | C1 | C2 | C3 |
| Hydroxypropyl | 0.15 | 0.15 | 0.15 |
| Methyl | | | |
| Cellulose | | | |
| Sodium Phosphate | 0.12 | 0.12 | 0.12 |
| dibasic, | | | |
| heptahydrate | | | |
| Sodium Phosphate | 0.01 | 0.01 | 0.01 |
| mnobasic, | | | |
| monohydrate | | | |
| Sodium Chloride | 0.55 | 0.55 | 0.55 |
| Potassium Chloride | 0.14 | 0.14 | 0.14 |
| Pluronic F87 | 0.05 | 0.05 | 0.05 |
| PropyleneGlycol | 0.5 | 0.5 | 0.5 |
| Taurine | 0 | 0.05 | 0.05 |
| EDTA | 0.01 | 0.01 | 0.01 |
| PHMB | 0.0001 | 0.0001 | 0.00014 |
| | (1.0 ppm) | (1.0 ppm) | (1.4 ppm) |
| PH | 7.41 | 7.39 | 7.41 |
| Osmolality | 283 | 288 | 287 |
| (mOsm/kg) | | | |

There were no significant differences between groups for burning/stinging, blurry vision, dry eye feeling, unusual eye secretions, increased lens awareness, redness or light sensitivity. Excessive tearing showed significant differences for C1 vs C2 (p=0.001), C1 vs. C3 (p=0.001) and C1 vs. O-F (p=0.001). Itching showed significant differences for C1 vs. C3 (p-0.049) and C1 vs. O-F (p=0.049). In all of the above comparisons, C1 was the least preferred by the test groups based on the tearing and itching caused.

Table 2 summarizes the incidence of itching among the four test groups.

TABLE 2

| | Solution | | | |
|---|---|---|---|---|
| Itching Symptom | C1 | Solution C2 | Solution C3 | Opti-Free |
| Moderate or severe, | 4.8% | 0% | 0% | 1.2% |
| day 90 | | | | |
| Moderate or severe, | 7.2% | 0% | 0% | 1.2% |
| overall | | | | |
| Mild, day 90 | 1.2% | 3.6% | 1.2% | 0.0% |
| Mild, overall | 4.8% | 8.4% | 4.8% | 1.2% |

It can be seen that within the carefully controlled C1-C3 series of nearly identical solutions, the inclusion of taurine in solutions C2 and C3 had a significant effect in eliminating the moderate or severe itching observed in the C1 solution which did not contain taurine. Itching was also monitored at the inception of the study (baseline) and on days 7, 30 and 60. Only solution C1 produced moderate or severe itching at days 7 and 30. It is also worth noting that excessive tearing was found in the C1 group at all intervals except at baseline. Such excessive tearing was not seen with C2 and C3.

### EXAMPLES 2-3

The following are given as examples of contact lens multi-purpose solutions according to the present invention, and are not intended to be limiting:

| | Example 2 | Example 3 |
|---|---|---|
| Ingredient | % w/v | % w/v |
| PHMB (ppm) | 1.1 | 1.1 |
| EDTA | 0.01 | 0.01 |
| HPMC | 0.15 | 0.15 |
| Propylene Glycol | 0.5 | 0.5 |
| NaCl | 0.55 | 0.55 |
| KCI | 0.14 | 0.14 |
| Dibasic Sodium | 0.12 | 0.12 |
| Phosphate 7H2O | | |
| Monobasic Sodium | 0.01 | 0.01 |
| Phosphate H2O | | |
| Pluronic F87 | 0.05 | 0.05 |
| Taurine | 0.05 | 0.20 |
| pH adjust w/Sodium | 7.4 | 7.4 |
| Hydroxide or HCI | | |
| Purified Water | q.s. 100 | q.s. 100 |

The solutions according to example 2 and 3 may be used, for example, to clean contact lenses. In this embodiment of the invention, approximately three (3) ml of this solution is introduced into a lens vial containing a lipid, oily deposit laden, hydrophilic or soft contact lens. The contact lens is maintained in this solution at room temperature for at least about four (4) hours. This treatment is effective to disinfect the contact lens. In addition, it is found that a substantial portion of the deposits previously present on the lens has been removed. This demonstrates that this solution has substantial passive contact lens cleaning ability. Passive cleaning refers to the cleaning which occurs during soaking of a contact lens, without mechanical or enzymatic enhancement.

After this time, the lens is removed from the solution and is placed in the lens wearer's eye for safe and comfortable wear. Alternately, after the lens is removed from the solution, it is rinsed with another quantity of this solution and the rinsed lens is then placed in the lens wearer's eye for safe and comfortable wear.

### EXAMPLES 4-7

The following examples can be used as contact lens rewetters:

| | Example 4 | Example 5 | Example 6 | Example 7 |
|---|---|---|---|---|
| Ingredient | % w/w | % w/w | % w/w | % w/w |
| Boric Acid | 0.6 | 0.6 | 0.6 | 0.6 |
| Sodium Borate 10 H20 | 0.035 | 0.035 | 0.035 | 0.035 |
| CaC12.2 H20 | 0.006 | 0.006 | 0.006 | 0.006 |
| MgC12.6 H20 | 0.006 | 0.006 | 0.006 | 0.006 |
| KCI | 0.14 | 0.14 | 0.14 | 0.14 |
| NaCl | 0.25 | 0.25 | | |
| Glycerin | | | 1 | 1 |
| HPMC | | 0.1 | 0.1 | |
| PHMB (ppm) | 0.6 | 0.6 | 0.6 | 0.6 |
| Taurine | 0.05 | 0.20 | 0.50 | 0.05 |
| pH | 7.25 | 7.25 | 7.25 | 7.25 |
| Purified Water | q.s. 100 | q.s. 100 | q.s. 100 | q.s. 100 |

The solutions according to examples 4-7 may be used, for example, to wet or rewet contact lenses. A hydrophilic contact lens is ready for wear. In order to facilitate such wearing, one or two drops of one of the solutions of Examples 4-5 is placed on the lens immediately prior to placing the lens in the lens wearer's eye. The wearing of this lens is comfortable and safe.

Alternatively, a lens wearer wearing a contact lens may apply one or two drops of one of the solutions of Examples 4-5 in the eye wearing the lens. This effects a re-wetting of the lens and provides for comfortable and safe lens wear.

While this invention has been described with respect to various specific examples and embodiments, it is to be understood that the invention is not limited thereto and that it can be variously practiced within the scope of the following claims.

What is claimed is:

1. A multi-purpose solution comprising:
an aqueous liquid medium;
an antimicrobial component in an amount effective to disinfect a contact lens contacted with said solution;
taurine in an amount effective to protect ocular tissue cell membranes;
a surfactant in an amount effective in cleaning a contact lens contacted with said solution; and
a phosphate buffer component in an amount effective in maintaining the pH of said solution within a physiologically acceptable range.

2. The solution as in claim 1, further comprising a viscosity inducing component selected from the group consisting of cellulosic derivatives and mixtures thereof in the range of about 0.05% to about 5.0% (w/v) of the total solution.

3. The solution as in claim 1, further comprising a chelating component in an amount of less than 0.05% (w/v) of the total solution.

4. The solution as in claim 1, further comprising a tonicity component in an amount effective in providing the desired tonicity to the solution.

5. A multi-purpose solution for contact lens care comprising:
an aqueous liquid medium;
an antimicrobial component in an amount effective to disinfect a contact lens contacted with said solution;
taurine in an amount effective to protect ocular tissue cell membranes;
a surfactant in an amount effective in cleaning a contact lens contacted with said solution;
a phosphate buffer component in an amount effective in maintaining the pH of said solution within a physiologically acceptable range;
a viscosity inducing component selected from the group consisting of cellulosic derivatives and mixtures thereof in the range of about 0.05% to about 5.0% (w/v) of the total solution;
a chelating component in an amount of less than 0.05% (w/v) of the total solution; and
a tonicity component in an amount effective in providing the desired tonicity to said solution.

6. The multi-purpose solution of claim 5, wherein the antimicrobial component is selected from the group consisting of biguanides, biguanide polymers, monomeric quaternary ammonium compound, salts thereof and mixtures thereof.

7. The multi-purpose solution of claim 5, wherein the antimicrobial component is present in an amount ranging from about 0.1 ppm to about 3 ppm.

8. The multi-purpose solution of claim 5, wherein the surfactant is selected from the group consisting of poly(oxyethylene)-poly(oxypropylene) block copolymers and mixtures thereof, and is present in an amount in a range of about 0.01% to about 1.0% (w/v).

9. The multi-purpose solution of claim 5, wherein the surfactant is present in an amount in the range of about 0.01% to about 1.0% (w/v).

10. The multi-purpose solution of claim 5, wherein the phosphate buffer component includes a combination of sodium hydrogen phosphate and sodium dihydrogen phosphate.

11. The multi-purpose solution of claim 5, wherein the phosphate buffer component is present in an amount in a range of about 0.01% to about 0.5% (w/v).

12. The multi-purpose solution of claim 5, wherein the viscosity inducing component is hydroxypropylmethyl cellulose.

13. The multi-purpose solution of claim 5, wherein the tonicity component includes a combination of sodium chloride and potassium chloride and is present in a range of about 0.4% to about 1.5% (w/v).

14. The multi-purpose solution of claim 5, wherein the chelating component is EDTA.

15. A method for maintaining ocular tissue cell membrane integrity during contact lens wear comprising contacting the lens with an isotonic aqueous solution comprising from about 0.1 ppm to about 100 ppm of the total solution of an antimicrobial component and from about 0.01% to about 2% w/v of the total solution of taurine.

16. The method for disinfecting of claim 15, wherein the isotonic solution further comprises a component selected from the group consisting of a viscosity inducing agent, a chelating agent and a tonicity component.

17. A method for maintaining ocular tissue cell membrane integrity during contact lens wear comprising contacting a lens positioned in a user's eye with an isotonic aqueous solution comprising:
an aqueous liquid medium;
an antimicrobial component in an amount effective to disinfect a contact lens contacted with said solution;
taurine in an amount effective to protect ocular tissue cell membranes;
a surfactant in an amount effective in cleaning a contact lens contacted with said solution; and;
a phosphate buffer component in an amount effective to maintain the pH of said solution within a physiologically acceptable range.

18. A process for mitigating ocular tissue insult comprising:
administering an ophthalmically neutral or beneficial solution to a user's eye where said solution further comprises taurine in an amount effective to protect ocular tissue cell membranes.

19. The process of claim 18, further comprising the step of contacting a user's eye with an aqueous liquid medium designed for temporary emplacement in the user's eye or allowing sufficient time for uptake of the ophthalmically neutral or beneficial solution into at least one of a soft-contact lens and a rigid gas permeable lens.

20. The process of claim 18, wherein administering step is conducted so that the aqueous liquid medium is temporarily emplaced in the user's eye.

21. The process of claim 18, wherein administering step is conducted so that uptake of the aqueous liquid medium into at least one of a soft-contact lens and a rigid gas permeable lens is achieved.

22. A method for maintaining ocular tissue cell membrane integrity during contact lens wear comprising contacting the lens with a hypotonic aqueous solution comprising from about 0.1 ppm to about 100 ppm of the total solution of an antimicrobial component and from about 0.01% to about 2% w/v of the total solution of taurine.

23. The method for disinfecting of claim 22, wherein the hypotonic solution further comprises a component selected from the group consisting of a viscosity inducing agent, a chelating agent and a tonicity component.

24. A method for maintaining ocular tissue cell membrane integrity during contact lens wear comprising contacting a lens positioned in a user's eye with a hypotonic aqueous solution comprising:
an aqueous liquid medium;
an antimicrobial component in an amount effective to disinfect a contact lens contacted with said solution;
taurine in an amount effective to protect ocular tissue cell membranes;
a surfactant in an amount effective in cleaning a contact lens contacted with said solution; and a phosphate buffer component in an amount effective in maintaining the pH of said solution within a physiologically acceptable range.
